(19) United States
(12) Patent Application Publication
(10) Pub. No.: US 2010/0081970 A1
(43) Pub. Date: Apr. 1, 2010

(54) Apparatus for improving health

(76) Inventor: Hsin Hsiang Pan, Taipei (TW)

Correspondence Address: SAM CHEN, 7F-1, 293, ROOSEVELT ROAD, SEC 3, TAIPEI, TW

(21) Appl. No.: 12/242,930

(22) Filed: Oct. 1, 2008

(51) Int. Cl. A61N 7/00; A61H 1/00

(52) U.S. Cl. 601/2; 601/15

(57) ABSTRACT

An apparatus for improving health includes a therapeutic assembly comprising a control panel, a horizontal central channel, a hollow cylinder rotatably disposed in the channel, a sound reproducing device, a light source adapted to emit colorful light rays, a pulsed low frequency generating unit adapted to generate magnetic resonance, an IR generator, a nano lamp, and an electromagnetic wave generating unit; and a table having a top magnetic massaging bed adapted to move lengthwise with respect to the table to dispose in the cylinder, the magnetic massaging bed being adapted to generate static magnetic fields.

### BACKGROUND OF THE INVENTION

1. Field of Invention

The invention relates to electromagnetic devices for alternative medicine and more particularly to an apparatus for improving health by utilizing static magnetic fields, sonic energy, electromagnetic field, and optical waves.

2. Description of Related Art

Effect of magnetic therapy is known by many people throughout the world. These types of therapeutic magnetic pulser device are said to be capable of releasing pain and/or even treating disease by subjecting certain parts of the body to static magnetic fields.

There have been numerous suggestions in prior patents for alternative medicine. For example, U.S. Pat. No. 4,665,898 discloses an apparatus which claims to eliminate tumor. Thus, continuing improvements in the exploitation of apparatus for improving health are constantly being sought.

### SUMMARY OF THE INVENTION

It is therefore one object of the invention to provide an apparatus for improving health by utilizing static magnetic fields, sonic energy, electromagnetic field, and optical waves.

The above and other objects, features and advantages of the invention will become apparent from the following detailed description taken with the accompanying drawings.

### BRIEF DESCRIPTION OF THE DRAWINGS

FIG. 1 is a perspective view of a preferred embodiment of apparatus for improving health according to the invention;

FIG. 2 is a schematic top view of FIG. 1; and

FIG. 3 is a side elevation of FIG. 1.

### DETAILED DESCRIPTION OF THE INVENTION

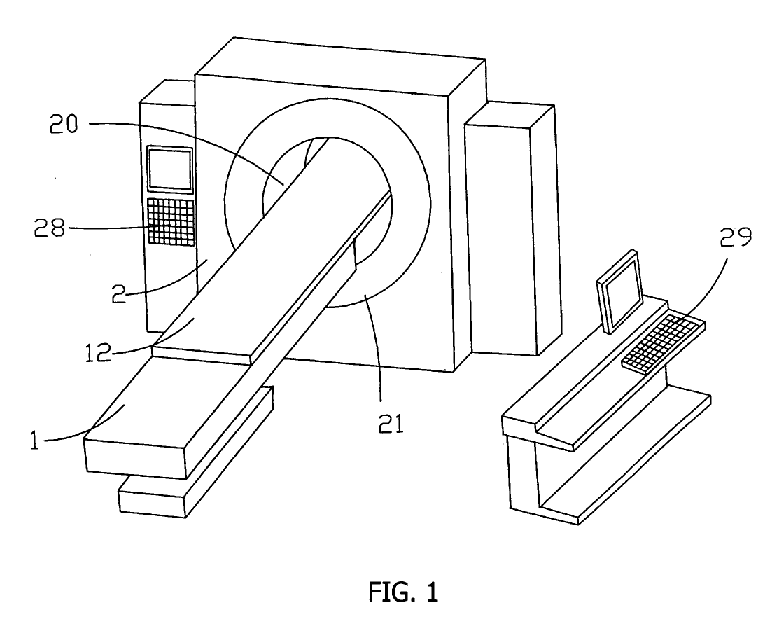

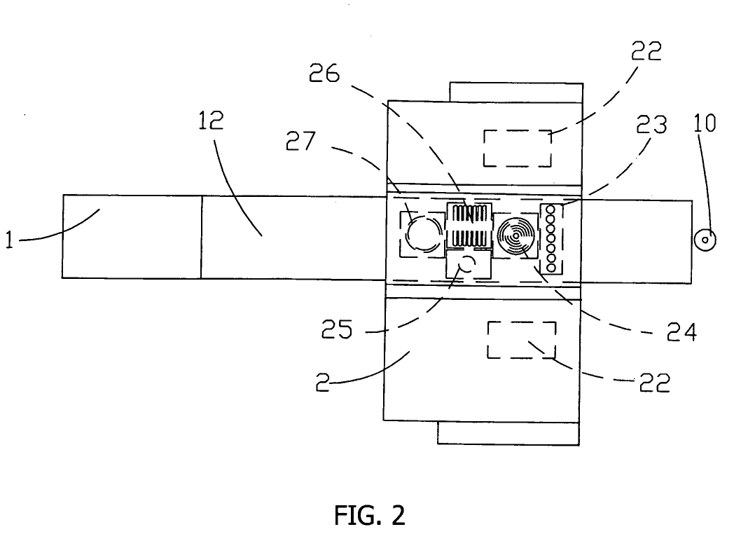

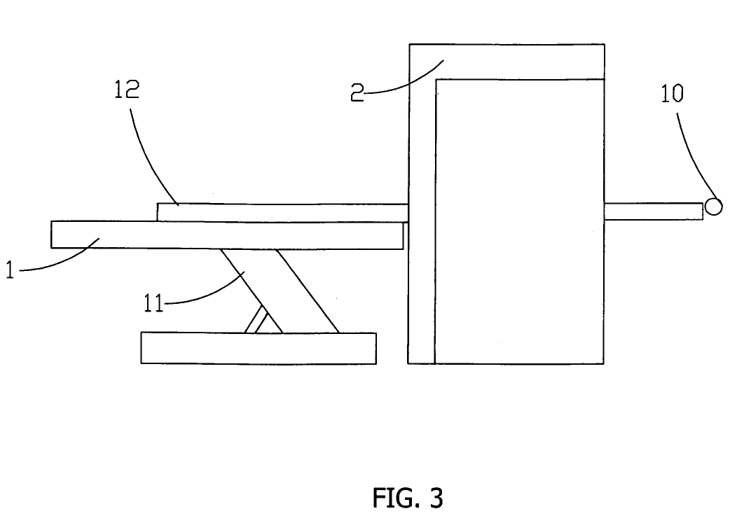

Referring to FIGS. 1 to 3, an apparatus for improving health in accordance with a preferred embodiment of the invention is shown. The apparatus comprises the following components as discussed in detail below.

A table 1 comprises a height adjustable base 11 for supporting the table 1, a magnetic massaging bed 12 adapted to move lengthwise with respect to the table 1, and an oxygen supplying device 10 at the other end of the magnetic massaging bed 12 distal the base 11. The magnetic massaging bed 12 is adapted to generate static magnetic fields for releasing pain of a person lying thereon.

A therapeutic assembly 2 is a rectangular body having two side projections (not numbered) of also rectangular body. The therapeutic assembly 2 comprises a control panel 28 provided on the left projection (see FIG. 1). Additionally, the therapeutic assembly 2 comprises a remote control panel 29 so that an operator may operate the apparatus from a remote site.

The therapeutic assembly 2 further comprises a horizontal central channel 20 of circular section cut along the vertical direction, the magnetic massaging bed 12 being adapted to moveably pass through the channel 20 so as to place a person lying thereon into the channel 20, a rotatable hollow cylinder 21 provided on the circumferential surface of the channel 20, and a sound reproducing device (not shown) having two opposite speakers 22 provided in the therapeutic assembly 2 spaced from both sides of the cylinder 21 respectively, the speakers 22 being in audio communication with the magnetic massaging bed 12 through the cylinder 21.

The following components of the therapeutic assembly 2 are provided above the top of the cylinder 21. A light source 23 is adapted to emit and impinge colorful light rays of about 480 μm (micrometer) to 3,400 μm on the body of a person lying on the magnetic massaging bed 12 which is disposed in the cylinder 21 by moving. Preferably, heat of about 40 mW/cm² is generated at a distance of about 20 cm from the skin. Hence, the skin temperature will not increase above 40° C. A pulsed low frequency generating unit 24 is provided in front of the light source 23 for generating magnetic resonance. An IR (infra-red) generator 25 is provided in front of the pulsed low frequency generating unit 24 and is adapted to generate IR waves having a wavelength of about 5 μm to 25 μm and impinge same on the body. A nano lamp 26 is provided besides the IR generator 25. The nano lamp 26 is adapted to emit light rays having a wavelength of about 9,100 nm (nanometer) to 9,900 nm and impinge same on the body. An electromagnetic wave generating unit 27 is provided in front of both the IR generator 25 and the nano lamp 26. The electromagnetic wave generating unit 27 is adapted to generate electromagnetic waves having a low frequency of about 8.6 Hz to 73 Hz and impinge same on the body.

Light rays emitted by the light source 23 are without the component of UV (ultra-violet). Preferably, the light source is comprised of a lens, a color filter, and a halogens lamp.

Preferably, the sound reproducing device is adapted to play easy listening music or classical music so as to bring a degree of comfort to the person lying on the magnetic massaging bed 12.

Preferably, the pulsed low frequency generating unit 24 is pulsed at an interval of 0.5 ms and is adapted to generate frequencies of 0.5 Hz, 1 Hz, 2 Hz, 5 Hz, 10 Hz, 20 Hz, 30 Hz, 70 Hz, and 100 Hz in a continuous mode or an intermittent mode during a time span of from 20 minutes to 30 minutes.

Preferably, the nano lamp 26 is formed of nano material and preferably may operate in a time length of 30 minutes to one hour. The light emitted by the nano lamp 26 may have many beneficial health effects.

Note that the cylinder 21 rotates continuously during the operation of the apparatus.

It is envisaged by the invention that static magnetic fields, sonic energy, electromagnetic field, and optical waves are utilized so as to bring beneficial health effects to the various parts of the body.

While the invention herein disclosed has been described by means of specific embodiments, numerous modifications and variations could be made thereto by those skilled in the art without departing from the scope and spirit of the invention set forth in the claims.

What is claimed is:

1. An apparatus for improving health comprising:
a therapeutic assembly comprising a first control panel, a horizontal central channel, a hollow cylinder rotatably disposed in the channel, a sound reproducing device spaced from the cylinder and being in audio communication with an internal space defined by the cylinder, a light source adapted to emit colorful light rays, a pulsed low frequency generating unit adapted to generate magnetic resonance, an IR (infra-red) generator adapted to generate IR waves, a nano lamp adapted to emit light rays of a predetermined wavelength, and an electromagnetic wave generating unit adapted to generate electromagnetic waves of a predetermined frequency; and
a table having a top magnetic massaging bed adapted to move lengthwise with respect to the table to dispose in the cylinder, the magnetic massaging bed being adapted to generate static magnetic fields.

2. The apparatus of claim 1, further comprising an oxygen supplying device at the other end of the magnetic massaging bed distal the table.

3. The apparatus of claim 1, wherein the therapeutic assembly further comprises a second control panel disposed from a predetermined remote site.

4. The apparatus of claim 1, wherein the IR generator is adapted to generate IR waves having a wavelength of about 5 μm to 25 μm.

5. The apparatus of claim 1, wherein the predetermined wavelength of the light rays emitted by the nano lamp is about 9,100 nm to 9,900 nm.

6. The apparatus of claim 1, wherein the predetermined frequency of the electromagnetic waves generated by the electromagnetic wave generating unit is about 8.6 Hz to 73 Hz.
